(19) United States
(12) Patent Application Publication
(10) Pub. No.: US 2013/0071300 A1
(43) Pub. Date: Mar. 21, 2013

(54) BILLS-ONLY STERILIZER

(71) Applicant: Jong-Yun LEE, Cheonan-si (KR)

(72) Inventor: Jong-Yun LEE, Cheonan-si (KR)

(21) Appl. No.: 13/616,491

(22) Filed: Sep. 14, 2012

(30) Foreign Application Priority Data
Sep. 15, 2011 (KR) 10-2011-0093089

(51) Int. Cl. A61L 2/08; A61L 2/16

(52) U.S. Cl. 422/291; 250/492.1

(57) ABSTRACT

The present invention generally relates to a bills-only sterilizer capable of sterilizing a little bills, and more particularly, to a bills-only sterilizer that can cause the bills under sterilization to be spaced more efficiently and enhance the efficiency of sterilization maximally. The bills-only sterilizer comprises a door opened and closed to insert bills into main body case and pull bills out of the main body case, a sterilizing chamber for sterilizing bills, the sterilizing chamber including a holding means and a sterilizing means, the holding means capable of holding bills therein when the door is opened, a blowing means under the sterilizing chamber, the blooding means blowing wind upwards, thereby causing bills to be spaced, an exhausting means formed back face of the main body case, the exhausting means connected to the sterilizing chamber and the exhausting means going through to the outside of the main body case.

### CROSS-REFERENCE TO RELATED APPLICATION

This application claims the benefit of Korean Patent Application No. 10-2011-0093089 filed on Sep. 15, 2011, which is herein incorporated by reference as if fully set forth herein.

### BACKGROUND OF THE INVENTION

The present invention generally relates to a bills-only sterilizer capable of sterilizing a little bills, and more particularly, to a bills-only sterilizer that can cause the bills under sterilization to be spaced more efficiently and enhance the efficiency of sterilization maximally.

Generally, it takes more than about four years that withdraw to discard bills after they are distributed availably. In spite of such as circulation of bills for a long time, bills are circulated without any sterilizing process.

In case of the circulation of bills without any sterilizing process, as is well known, bills are contaminated by various pathogenic micro-organizations such as bacteria and mold etc., thereby the bills serve as a medium causing allergic ailments such as various skin ailments and respiratory ailments etc. or infecting people. In particular, it is likely that the contaminated bills have great damage to not only the old and the infirm having weak immunity but also a person in charge of dealing in bills in banking facilities. Nevertheless, there is no alternative.

In order to solve this problem, KR patent application No. 2001-0009068 “The antiseptic method and equipment of the Bank bills by the antibiotic natural essential oil from the herb” is proposed, but since 2001-0009068 put and sterilize an antiseptic into sterilizing chamber in condition of vacuum and with spray atomizing type using gas compressed in high-pressure and nozzles, this not only have the complex structure of additional facilities due to using the compressed gas, but also costs high thereby. Moreover, there is restriction on installation space since it is high capacity for sterilization of much bills

KR patent No. 10-0568012 “The antiseptic method and equipment of the Bank bills by the antibiotic natural essential oil from the herb” disclosed a method putting an antiseptic into a sterilizing chamber in a vacuum with employing a putting method using a natural vacuum pressure, wherein the antiseptic is prepared by using herb refining having an excellent antibacterial and antifungal function as a main material.

### SUMMARY OF THE INVENTION

However, the method and equipment of the above patent are restricted in installation conditions since they are used for sterilizing a large quantity of bills. Moreover, they have facilities for maintaining a vacuum state and a complex structure required in spraying the antiseptic. Therefore, there is a problem that they are suitable for separate sterilizing facilities, not for using them respectively.

Furthermore, since the sterilizing method in the above patent application or the above patent spray an antiseptic while loading bills, the antiseptic is not properly put between bills, thereby the efficiency of sterilization is not good.

Therefore, the present invention is devised to solve the above problems, an object of the present invention is to provide a bills-only sterilizer that can cause the bills under sterilization to be spaced more efficiently and enhance the efficiency of sterilization maximally.

Another object of the present invention is to minimize interference according to and closing a door.

Another object of the present invention is to hold bills under sterilization stably and easily.

Another object of the present invention is to check the sterilizing process of bills with ease externally.

Another object of the present invention is to blow wind between bills effectively.

Another object of the present invention is to maximize the efficiency of sterilization.

According to an aspect of the present invention, a bills-only sterilizer for sterilizing bills is provided, the bills-only sterilizer comprising: a door opened and closed to insert bills into main body case and pull bills out of said main body case; a sterilizing chamber for sterilizing bills, said sterilizing chamber including a holding means and a sterilizing means, said holding means capable of holding bills therein when said door is opened; a blowing means under said sterilizing chamber, said blowing means blowing wind upwards, thereby causing bills to be spaced; an exhausting means formed back face of said main body case, said exhausting means connected to said sterilizing chamber, and said exhausting means going through to the outside of said main body case, wherein said holding means including a back face supporting plate, said back face supporting plate formed rearward of said sterilizing chamber, a supporting bracket, said supporting bracket formed to support both sides of the bottom of bills inserted into both sides of a bottom face, a guide rail, said guide rail located over said supporting bracket and supporting the left and the right of said inserted bills, front supporting panel, said front supporting panel inserted on said guide rail to slide back and forth and supporting the front of said inserted bills, and a top panel, said top panel installed to be lifted and lowered freely over the top of said back face supporting plate and supporting the top of said inserted bills.

Advantageously, said front supporting panel is formed from a transparent material, and said front supporting panel further comprises a perforation perforating the center of said from supporting panel.

Advantageously, said sterilizing means is one or more than one sterilizing lamp, said one or more than one sterilizing lamp installed in one side or both side of said sterilizing chamber.

Advantageously, said blowing means comprise a plurality of blowing holes and a blower, said blowing holes formed by perforating bottom face of said sterilizing chamber, the entire area of said blowing holes corresponding to that of said holding means.

Advantageously, said bills-only sterilizer further comprise a sterilizing unit, said sterilizing unit located under the sterilizing chamber and said sterilizing unit releasing a sterilizing solution into said sterilizing chamber.

The present invention can cause the bills under sterilization to be spaced more efficiently and enhance the efficiency of sterilization maximally, thereby can use bills in a clean state by sterilizing quickly and conveniently if necessary.

Moreover, the present invention can enhance the convenience of use since a door is opened and closed up and down, thereby interference with other articles can be minimized at its installation place.

Moreover, the present invention can hold bills under sterilization stably and easily, thereby sterilization of bills can be performed more effectively and more cleanly.

Moreover, the sterilizing process of bills can be checked with ease externally, thereby a user can see whether the sterilization is processing or not with naked eye, and reliability can increase.

Moreover, the present invention can blow wind between bills effectively, thereby the efficiency of sterilization can be enhanced.

Moreover, the bills-only sterilizer according to the present invention includes one sterilizing unit as well as a sterilizing lamp, thereby the sterilization can be processed in a maximum clean state.

### BRIEF DESCRIPTION OF THE DRAWINGS

FIG. 1 is a perspective figure of main part representing schematically the present invention.

FIG. 2 is a figure of main part representing inner structure where a door is opened in the embodiment of FIG. 1.

FIG. 3 is a side section of main part according to FIG. 2

FIG. 4 is a figure of main part representing schematically the state implementing the present invention.

FIG. 5 is a figure of main part representing schematically another embodiment according to the present invention.

### DETAILED DESCRIPTION OF THE INVENTION

In the following detailed description, the most desirable embodiments of the present disclosure are described in detail, with reference to attached figures, in order to enable an ordinary person skilled in the art to implement the present disclosure.

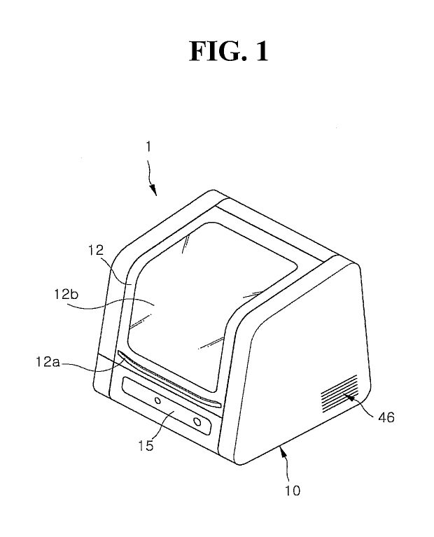

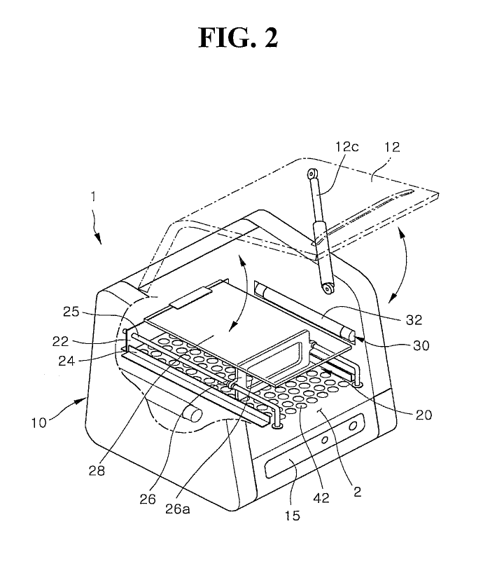

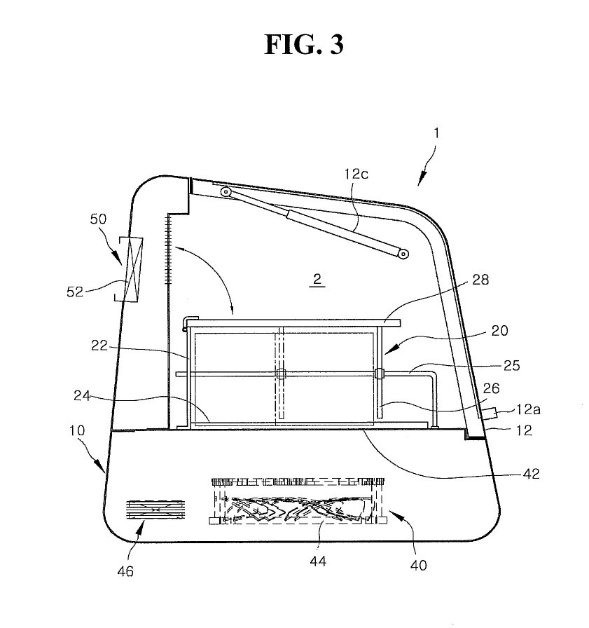

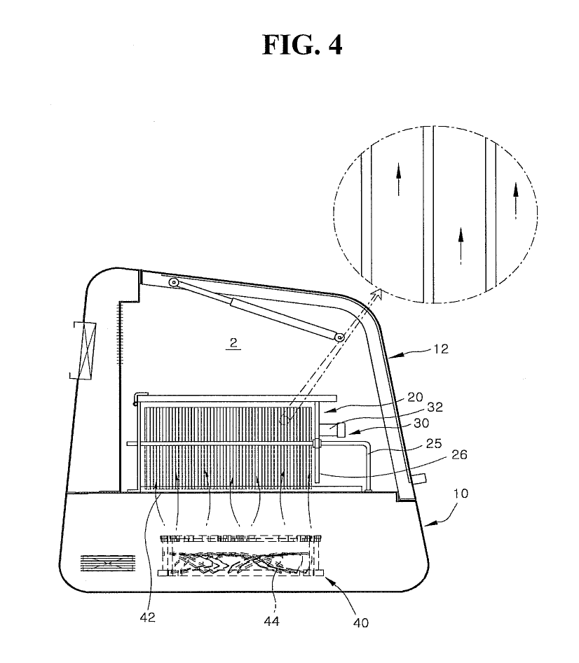

FIG. 1 is a perspective figure of main part representing schematically the present invention, FIG. 2 is a figure of main part representing inner structure where a door is opened in the embodiment of FIG. 1, FIG. 3 is a side section of main part according to FIG. 2, and FIG. 4 is a figure of main part representing schematically the state implementing the present invention.

As described in FIGs, bills-only sterilizer to sterilize bills polluted after circulated commercially is provided. In order to cause the bills under sterilization to be spaced more efficiently and enhance the efficiency of sterilization maximally, the bills-only sterilizer according to the present invention comprises, a door(12) opened and closed to insert bills into main body case(10) and pull bills out of said main body case(10); a sterilizing chamber(2) for sterilizing bills, said sterilizing chamber including a holding means(20) and a sterilizing means(30), said holding means capable of holding bills therein when said door(12) is opened; a blowing means(40) under said sterilizing chamber(2), said blowing means blowing wind upwards, thereby causing bills to be spaced; an exhausting means(50) formed back face of said main body case, said exhausting means connected to said sterilizing chamber, and said exhausting means going through to the outside of said main body case.

A reference number 15(not described in the above description) is an operating panel including a well-known control means, wherein the control means can operate or stop with said sterilizing means or said blowing means and said exhausting means.

Advantageously, the exhausting means(50) may include a well-known separate exhausting fan in the back face of the main body case(10). Furthermore, the exhausting means(50) may also include a filter(52) which is attached or removed in a conventional way if necessary, thereby harmful materials included in the air after bills are sterilized in the sterilizing chamber can be filtered.

Advantageously, the door(12) may include a grip(21a) formed on its outer and a perspective unit(12b) made by a transparent material, wherein the grip(21a) enable user to griping easily when open or close the door(12), and the perspective unit(12b). The door(12) can be opened or closed up and down by a conventional damper(12c), wherein the damper(12c) is installed within the inner of the sterilizing chamber. Thus, interference with other articles according to left and light opening or closing of the door(12) can be reduced, and the sterilizing process within the sterilizing chamber can be seen with ease externally through the perspective unit as well.

Advantageously, the holding means(20) may include a back face supporting plate(22) which is formed rearward of the sterilizing chamber, a supporting bracket(24) which is formed to support both sides of the bottom of bills inserted into both sides of a bottom face, a guide rail(25) which is located over the supporting bracket and support the left and the right of the inserted bills, a front supporting panel(26) which is inserted on said guide rail to slide back and forth and support the front of the inserted bills, and a top panel(28) which is installed to be lifted and lowered freely over the top of the back face supporting plate by using a pin joint or a hinge joint and supports the top of the inserted bills. The top panel(28) is made of materials such as a transparent glass or acryl.

In other words, after inserting the bills under sterilization between the back supporting plate and the supporting bracket and the guide rail while lifting the tip panel(28) upwards, a user slides the front supporting panel to be located in front of the bills, and then lowers the top panel lifted. The holding means is therefore not only simple but also convenient for holding bills under sterilization.

That is, according to lift and lower the top panel(28) and slide the front supporting panel, a user can complete holding of bills under sterilization simply and conveniently without any inconvenience. In this regard, the front supporting panel may be slid to push such that the standing bills do not fall, in other words, in order that a space between the bills is kept loosely, not that the front supporting panel is not pushed to contact with a front of the bills, i.e., not that it is pushed closely to contact with the front of the bills.

Besides, it is advantageous that the front supporting panel is formed from a conventional glass or acryl. The front supporting panel also may be fixed by means of conventional bolts or screws after sliding in the guide rail.

The front supporting panel(26) is formed from a transparent material, and it further comprises a perforation(26a) perforating the center of the front supporting panel. Thus, the process of sterilization within the holding means can be seen easily through the perforation(26a) as well as the perspective unit. Furthermore, it can be also that any kind of bills are under sterilization through the perforation(26a).

The sterilizing means(30) is one or more than one sterilizing lamp(32) which is/are one or more than one well-known far-infrared lamp etc., wherein the sterilizing lamp(32) is installed in one side or both sides of the sterilizing chamber(2) by a conventional method. Thus, the bacteria or mold living in the bills is sterilized or the odor due to them is removed by the heating or drying with the sterilizing lamp(32). In this regard, the bills under sterilization is caused to be spaced by the blowing means, thereby the efficiency of sterilization can be enhanced.

Advantageously, The blowing means(40) comprises a plurality of blowing holes(42) and a blower(44), wherein the blowing holes(42) is formed by perforating bottom face of the sterilizing chamber, the entire area of the blowing holes corresponds to the entire area of the holding means, and the blower(44) blows under the blowing holes. In this regard, a inlet(46) such as a conventional inlet hole or slit groove is advantageously formed in a side or back face of the case comprising the blower, a conventional removable filter is advantageously included inside the inlet(46), wherein the filter can filter extraneous bodies included in the air flowed into the inlet.

According to the embodiment of the present invention, first, a user open the door(10) formed in the main body case(10), and then lift the top panel that is the holding means(20) within the sterilizing chamber(2), and then insert the bills under sterilization between the supporting bracket and the guide rail. At this time, the front supporting panel is located in the front of the guide rail, the bills is inserted under that state, and then the front supporting panel is slid and pushed to contact with the front of the bills and also lowered the top panel lifted upwards. Thus, the user completes holding the bills under sterilization by the holding means. In this regard, when push the front supporting panel to the front of the bills, the space between the bills is kept loosely such that the bills do not fall, not that it is pushed closely to contact with the front of the bills.

In this way, when completed holding the bills under sterilization, the user closes the door, and then operates the blowing means(40) and the sterilizing means(30), and then the wind blown by the blower(44) passes upwards the bottom of the bills under sterilization through the blowing holes(42). Thus, the space between the bills is spaced effectively by the wind blowing through the blowing holes from lower to upper. Therefore, the bills-only sterilizer according to the present invention has a high efficiency of sterilization by the sterilizing lamp(32) that is the sterilizing means(30). In other words, the space between the bills is spaced, thereby bacterium or molds living in the bills sterilized or the odor due to them is removed, thereby, the bills-only sterilizer according to the present invention has a high efficiency of sterilization.

When the sterilization is completed like this, the user opens the door and then slides the front supporting panel forward, and then pulls the bills between the supporting bracket and the guide rail. This process is in the opposite order of the inserting. Thus, a series of the sterilization is completed.

The present invention enables the space between the bills under sterilization to be spaced effectively by the wind blowing from lower to upper by blower and bacterium and molds to be removed living in the bills simultaneously. Therefore, the present invention has an advantage that can enhance the efficiency of sterilization of the bills under sterilization.

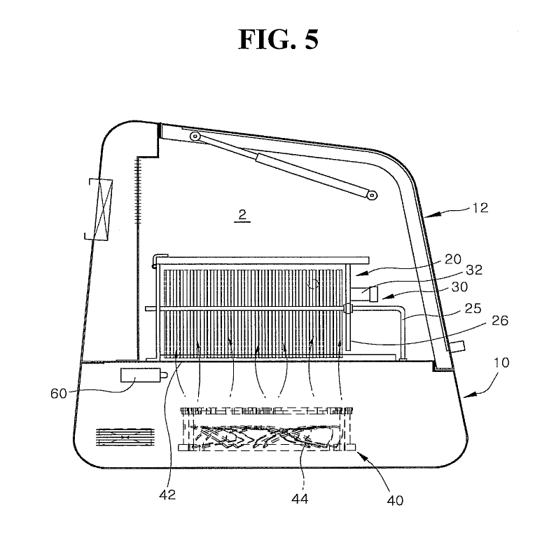

Meanwhile, as described in FIG. 5, in order to maximize the efficiency of sterilization during the sterilization, it is advantageous that the bills-only sterilizer further comprises a sterilizing unit(60) besides the sterilizing lamp, wherein the sterilizing lamp(60) is located under the sterilizing chamber(2) and releases a sterilizing solution into the sterilizing chamber(2).

The sterilizing unit(60) is to maximize the efficiency of sterilization together with the sterilizing lamp. In the sterilizing unit(60), a well-known emitter is installed. The sterilizing solution is sprayed into the sterilizing chamber through the blowing holes by the emitter, and then the sterilizing solution sprayed by the emitter circulates into the inside of the sterilizing chamber by the wind of the blower, thereby the wind is provided to the space between the bills. Therefore, the present invention can have an enhanced efficiency of sterilization more and more.

As stated above, the emitter can spray the sterilizing solution in a liquid state conventionally, and also employ the well-known principle of humidifier using ultrasonic waves or heating system, and also may employ a spray which scatters the sterilizing solution with a centrifugal force like the well-known principle of centrifugal humidifier and then enables the sterilizing solution to collide with a surrounding stationary vein to be atomized and then sprays them. In particular, when the sterilizing solution circulates into the sterilizing chamber in an atomized state and is sprayed between bills, the present invention can have more enhanced efficiency of sterilization.

1. A bills-only sterilizer for sterilizing bills, the bills-only sterilizer comprising:
a door opened and closed to insert bills into main body case and pull bills out of said main body case;
a sterilizing chamber for sterilizing bills, said sterilizing chamber including a holding means and a sterilizing means, said holding means capable of holding bills therein when said door is opened;
a blowing means under said sterilizing chamber, said blowing means blowing wind upwards, thereby causing bills to be spaced;
an exhausting means formed back face of said main body case, said exhausting means connected to said sterilizing chamber, and said exhausting means going through to the outside of said main body case,
wherein said holding means including a back face supporting plate, said back face supporting plate formed rearward of said sterilizing chamber, a supporting bracket, said supporting bracket formed to support both sides of the bottom of bills inserted into both sides of a bottom face, a guide rail, said guide rail located over said supporting bracket and supporting the left and the right of said inserted bills, front supporting panel, said front supporting panel inserted on said guide rail to slide back and forth and supporting the front of said inserted bills, and a top panel, said top panel installed to be lifted and lowered freely over the top of said back face supporting plate and supporting the top of said inserted bills.

2. The bills-only sterilizer of claim 1, wherein said front supporting panel is formed from a transparent material, and said front supporting panel further comprises a perforation perforating the center of said front supporting panel.

3. The bills-only sterilizer of claim 1, wherein said sterilizing means is one or more than one sterilizing lamp, said one or more than one sterilizing lamp installed in one side or both side of said sterilizing chamber.

4. The bills-only sterilizer of claim 1, wherein said blowing means comprises a plurality of blowing holes and a blower, said blowing holes formed by perforating bottom face of said sterilizing chamber, the entire area of said blowing holes corresponding to that of said holding means, and said blower blowing under the blowing holes.

5. The bills-only sterilizer of claim 4, wherein said bills-only sterilizer further comprises a sterilizing unit, said sterilizing unit located under the sterilizing chamber and said sterilizing unit releasing a sterilizing solution into said sterilizing chamber.
